(19) United States
(12) Patent Application Publication
(10) Pub. No.: US 2024/0294486 A1
(43) Pub. Date: Sep. 5, 2024

(54) GRISEOFULVIN 4 POSITION ETHERIFIED DERIVATIVES AND THEIR APPLICATION

(71) Applicant: Yan’an University, Yan'an (CN)

(72) Inventors: Yubin Bai, Yan'an (CN); Meng Zhang, Yan'an (CN); Liangzhu Huang, Yan'an (CN); Yu Zhao, Yan'an (CN)

(21) Appl. No.: 18/413,049

(22) Filed: Jan. 16, 2024

(30) Foreign Application Priority Data
Feb. 20, 2023 (CN) 2023101373785

(51) Int. Cl. C07D 307/94

(52) U.S. Cl. CPC C07D 307/94

(57) ABSTRACT

Provided are griseofulvin 4 position etherified derivatives and their application thereof. The chemical structures of the griseofulvin 4 position etherified derivatives are drawn in a formula I below. The application of these derivatives includes using the griseofulvin 4 position etherified derivatives as preventive or direct antifungal agents against phytopathogenic fungi.

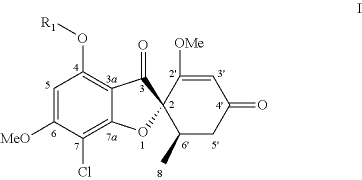

### TECHNICAL FIELD

The present disclosure is about griseofulvin 4 position etherified derivatives and their application, including griseofulvin 4 position etherified derivatives, synthetic procedure, and antifungal application against phytopathogenic fungi.

### BACKGROUND

Griseofulvin, a natural product, which was isolated from fungal mycelium of penicillium griseofulvum in 1939. Since it was isolated, hundreds of griseofulvin derivatives have been used for drug screening. (McBride, B. C. 1965, Doctoral dissertation, University of British Columbia.), It was used as an antifungal drug for dermatomycoses. (Arkley, V., Attenburrow, J., Gregory, G. I., & Walker, T. J. Chem. Soc. (Resumed). 1962, 1260-1268.).

In recent years, there are some new references reported that griseofulvin derivatives have antifungal activity against phytopathogenic fungi and some anticancer activity. (Petersen, A. B., Andersen, N. S., Konotop, G., Hanafiah, N. H. M., Raab, M. S., Krämer A., & Clausen, M. H. Eur. J. Med. Chem. 2017, 130, 240-247.; Bai, Y-B., Gao, Y-Q., Nie, X-D., Tuong, T.-M.-L., Li, D., Gao, J.-M. J. Agric. Food. Chem. 2019, 67(22), 6125-6132.).

### THE CONTENTS

A series of griseofulvin 4 position etherified derivatives are provided in the present disclosure. General chemical structures of griseofulvin 4 position etherified derivatives are shown in a formula I as below.

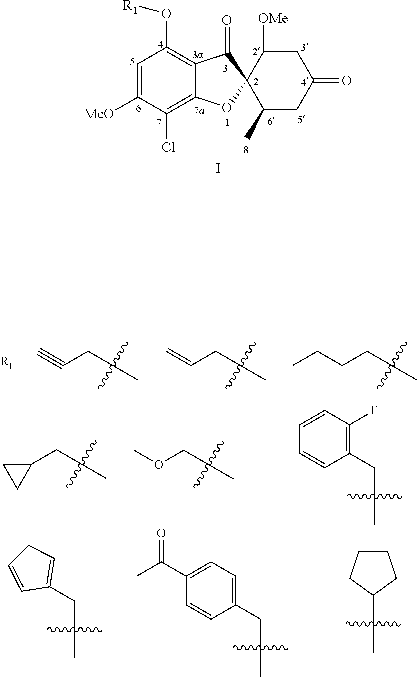

The preparation method of the synthesis of griseofulvin 4 position etherified derivatives is provided, which includes:
with organic solvent and base, intermediate II reacted with different bromoalkanes, griseofulvin 4 position etherified derivatives were obtained, a chemical structure of the intermediate II is shown below.

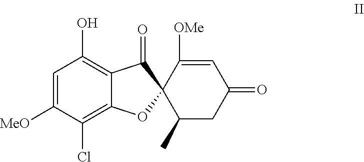 [SMILES: COC1=CC(=O)C[C@@H](C)[C@]12Oc1c(Cl)c(OC)cc(O)c1C2=O]

The appropriate methods could be used to obtain the desired product, for example the preparation method included but not limited that after the reaction finished, the reaction mixture was concentrated and purified by column chromatography in order, to get the desired product griseofulvin 4 position etherified derivatives.

Griseofulvin 4 position etherified derivatives can be used to be preventive or direct fungicide agents against phytopathogenic fungi in present disclosure. The phytopathogenic Fungi includes one or more of Cytospora sp., Alternaria solani Sorauer, Alternaria alternate, Botrytis sp., Botrytis cinerea Pers., Colletotrichum gloeosporioides, or Botryosphaeria dothidea.

### THE SPECIFIC PROCEDURE

The scientific and technical words in this document is based on the understanding of the common technicians in relative field, except where noted.

The chemical structures of griseofulvin derivatives (compound 01, 02, 03, 04, 05, 06, 07, 08 and 09) are drawn below.

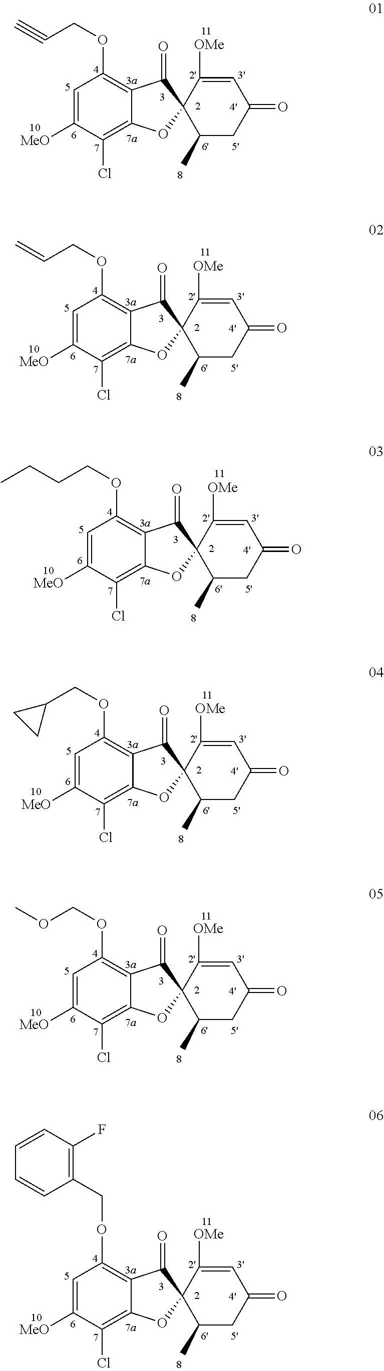 [SMILES: C#CCOc1cc(OC)c(Cl)c2c1C(=O)[C@@]1(O2)C(OC)=CC(=O)C[C@H]1C ; C=CCOc1cc(OC)c(Cl)c2c1C(=O)[C@@]1(O2)C(OC)=CC(=O)C[C@H]1C ; CCCCOc1cc(OC)c(Cl)c2c1C(=O)[C@@]1(O2)C(OC)=CC(=O)C[C@H]1C ; COC1=CC(=O)C[C@@H](C)[C@]12Oc1c(Cl)c(OC)cc(OCC3CC3)c1C2=O ; COCOc1cc(OC)c(Cl)c2c1C(=O)[C@@]1(O2)C(OC)=CC(=O)C[C@H]1C ; COC1=CC(=O)C[C@@H](C)[C@]12Oc1c(Cl)c(OC)cc(OCc3ccccc3F)c1C2=O]

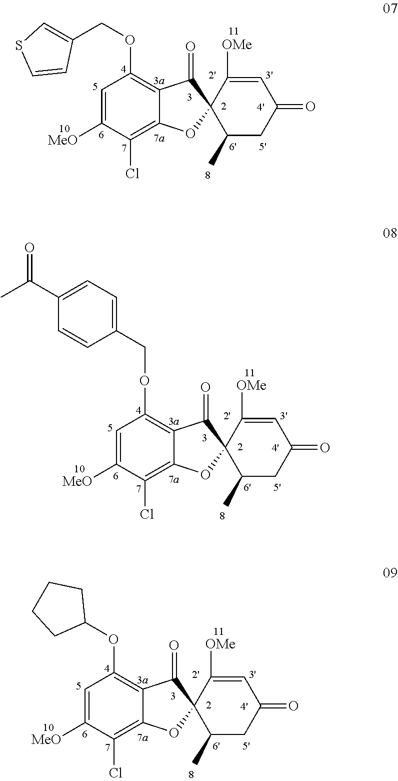 [SMILES: COC1=CC(=O)C[C@@H](C)[C@]12Oc1c(Cl)c(OC)cc(OCc3ccsc3)c1C2=O ; COC1=CC(=O)C[C@@H](C)[C@]12Oc1c(Cl)c(OC)cc(OCc3ccc(C(C)=O)cc3)c1C2=O ; COC1=CC(=O)C[C@@H](C)[C@]12Oc1c(Cl)c(OC)cc(OC3CCCC3)c1C2=O]

The synthetic route of griseofulvin 4 position etherified derivatives are shown below.

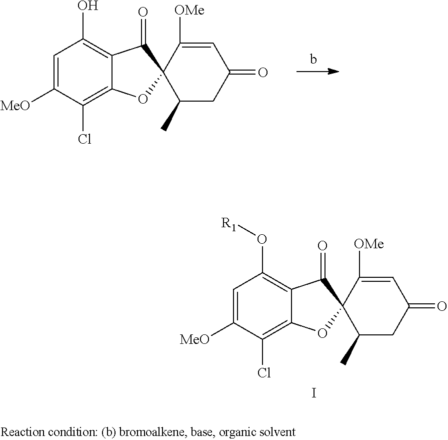

General procedure for the synthesis of griseofulvin 4 position etherified derivatives. With organic solvent and base, intermediate II reacted with different bromoalkanes, griseofulvin 4 position etherified derivatives were obtained. After reaction finished, the reaction mixture was concentrated and purified by column chromatography, the desired product griseofulvin 4 position etherified derivatives were obtained. In synthesis procedure, the researchers can choose appropriate organic solvent and base. The organic solvent was but was not limited to acetone and THE et al; The base could be, but was not limited to K₂CO₃, Na₂CO₃, Cs₂CO₃ et al.

In this work, intermediate II was obtained by MgI₂, which can selectively remove the methyl of griseofulvin at position 4, and chemical structure of intermediate II was shown as below.

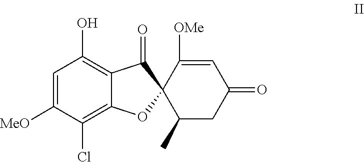 [SMILES: COC1=CC(=O)C[C@@H](C)[C@]12Oc1c(Cl)c(OC)cc(O)c1C2=O]

The synthesis procedure referenced a reported method (Bai Yu-Bin, Gao Yu-Qi, Nie Xiao-Di, Tuong Thi-Mai-Luong, Li Ding, Gao Jin-Ming. J. Agric. Food. Chem. 2019, 67 (22), 6125-6132.; Rønnest M H., Harris P., Gotfredsen C H., et al. Tetrahedron Letters. 2010, 51(45), 5881-5882.).

The procedure for the synthesis of intermediate II as follows.

The magnesium turnings (0.518 g, 21.6 mmol) and iodine (1.828 g, 7.2 mmol) were added to a solution of anhydrous Et₂O (4 mL) and toluene (8 mL) under argon. And the solution was refluxed at 80° C. for 30 min until the mixture turned to be a colorless solution, then the resulting solution was added to a solution of griseofulvin (1.411 g, 4 mmol) in dry toluene (10 mL), and heated to 80° C. for 4 h, H₂O (20 mL) and Na₂S₂O₃ were added, and the mixture was poured into 5% hydrochloric acid (10 mL) and extracted with EtOAc (3×20 mL), dried under MgSO₄, and concentrated. The residue was purified by column chromatography (CH₂Cl₂: MeOH: AcOH=97:2:1), a white solid was obtained and the yield is more than 80%.

The chemical structure of start material griseofulvin was shown below.

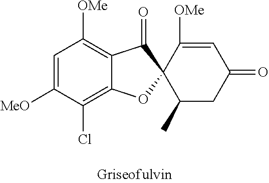 [SMILES: COC1=CC(=O)C[C@@H](C)[C@]12Oc1c(Cl)c(OC)cc(OC)c1C2=O]

The start material griseofulvin (Cas No., 126-07-8; purity, 97%) were purchased from commercial sources (Aladdin Reagent Company).

The examples of synthesis procedure of griseofulvin 4 position etherified derivatives were shown below. The temperature and concentration are approximate value. In specific procedure, technician could choose appropriate reagent ratio, concentration, temperature (room temperature to boiling point of the solvent), solvent, reagent adding order, heating method and purify method to obtain the desired products in this document.

### Example 1: The Synthesis Procedure of 4 Position Propargyl Griseofulvin Derivative (Compound 01)

The intermediate II (0.5 mmol) was dissolved in dry acetone (6 mL) solution at room temperature, then propargyl bromide (0.6 mmol) and K₂CO₃ (0.75 mmol) were added, reaction mixture were refluxed in oil bath, TLC monitored, after reaction finished, concentrated by rotavapor, and residue was purified by column chromatography on silica gel eluting with CH₂Cl₂: CH₃OH (40:1-100:1), white solid was obtained, yield 95%, m.p. 176-178° C. ¹H NMR (400 MHZ, CDCl₃) δ 6.37 (s, 1H, H-5), 5.54 (s, 1H, H-3′), 4.96-4.84 (m, 2H, —OCH₂—), 4.03 (s, 3H, H-10), 3.62 (s, 3H, H-11), 3.02 (dd, J=16.5, 13.5 Hz, 1H, H-5′), 2.89-2.79 (m, 1H, H-6′), 2.63 (s, 1H, CH≡C—), 2.43 (dd, J=16.6, 4.6 Hz, 1H, H-5′), 0.96 (d, J=6.7 Hz, 3H, H-8). ¹³C NMR (100 MHZ, CDCl₃) δ 197.17 (C-4′), 192.48 (C-3), 170.83 (C-2′), 169.65 (C-7a), 164.40 (C-6), 155.42 (C-4), 105.63 (C-3a), 105.02 (C-3′), 98.25 (C-7), 91.92 (C-5), 90.92 (C-2), 77.68 (CH≡C—), 77.08 (CH≡C—), 57.27 (—OCH₂—), 57.21 (C-10), 56.84 (C-11), 40.13 (C-5′), 36.57 in (C-6′), 14.38 (C-8). HR-MS (ESI): m/z calcd. for C₁₉H₁₇ClO₆H: 376.0714; found: 377.0792 [M+H]⁺.

Chemical structure of compound 01 was shown below.

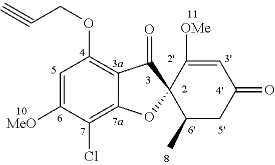 [SMILES: C#CCOc1cc(OC)c(Cl)c2c1C(=O)[C@@]1(O2)C(OC)=CC(=O)C[C@H]1C]

### Example 2: The Synthesis Procedure of 4 Position Allyl Griseofulvin Derivative (Compound 02)

The different in this procedure is that the bromoalkene is allyl bromide, and a white solid was obtained. Yield 72%, m.p. 200-202° C.; ¹H NMR (400 MHZ, CDCl₃) δ 6.14 (s, 1H, H-5), 6.11-6.01 (m, 1H, CH₂═CH—), 5.56-5.46 (m, 2H, H-3′, —CH—CH₂), 5.36 (dd, J=10.5, 1.4 Hz, 1H, —CH—CH₂), 4.71 (d, J=5.2 Hz, 2H, —OCH₂—), 3.99 (s, 3H, H-10), 3.61 (s, 3H, H-11), 3.03 (dd, J=16.7, 13.5 Hz, 1H, H-5′), 2.88-2.80 (m, 1H, H-6′), 2.44-2.39 (m, 1H, H-5′), 0.96 (d, J=6.7 Hz, 3H, H-8). ¹³C NMR (100 MHZ, CDCl₃) δ 197.20 (C-4′), 192.34 (C-3), 170.97 (C-2′), 169.57 (C-7a), 164.47 (C-6), 156.85 (C-4), 131.74 (CH₂═CH—), 118.94 (CH₂═CH—), 105.45 (C-3a), 104.91 (C-3′), 97.34 (C-7), 91.02 (C-5), 90.81 (C-2), 70.17 (—OCH₂—), 57.08 (C-10), 56.80 (C-11), 40.11 (C-5′), 36.58 (C-6′), 14.35 (C-8). HR-MS (ESI): m/z calcd. for C₁₉H₁₉ClO₆Na: 401.0768; found: 401.0779 [M+Na]⁺.

Chemical structure of compound 02 was shown below.

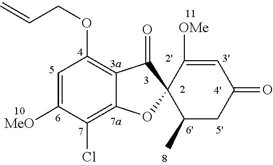 [SMILES: C=CCOc1cc(OC)c(Cl)c2c1C(=O)[C@@]1(O2)C(OC)=CC(=O)C[C@H]1C]

### Example 3: The Synthesis Procedure of 4 Position Butyl Griseofulvin Derivative (Compound 03)

The different in this procedure is that the bromoalkene is butyl bromide, and a white solid was obtained. Yield 86%, m.p. 145-146° C.; ¹H NMR (400 MHZ, CDCl₃) δ 6.11 (s, 1H, H-5), 5.53 (s, 1H, H-3′), 4.16-4.06 (m, 2H, —OCH₂—), 4.00 (s, 3H, H-10), 3.61 (s, 3H, H-11), 3.05 (dd, J-16.7, 13.5 Hz, 1H, H-5′), 2.86-2.80 (m, 1H, H-6′), 2.41 (dd, J=16.7, 4.7 Hz, 1H, H-5′), 1.90-1.83 (m, 2H, —CH₂—), 1.57-1.48 (m, 2H, —CH₂—), 1.05-0.90 (m, 6H, H-8, —CH₃). ¹³C NMR (100 MHz, CDCl₃) δ 197.23 (C-4′), 192.25 (C-3), 171.08 (C-2′), 169.58 (C-7a), 164.55 (C-6), 157.62 (C-4), 105.32 (C-3a), 104.90 (C-3′), 96.95 (C-7), 90.75 (C-5), 90.38 (C-2), 69.34 (—OCH₂—), 57.08 (C-10), 56.79 (C-11), 40.13 (C-5′), 36.63 (C-6′), 30.90 (—CH₂—), 19.21 (—CH₂—), 14.37 (C-8), 13.95 (—CH₃). HR-MS (ESI): m/z calcd. for C₂₀H₂₃ClO₆Na: 417.1081; found: 417.1086 [M+Na]⁺.

Chemical structure of compound 03 was shown below.

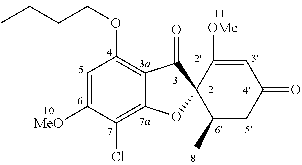 [SMILES: CCCCOc1cc(OC)c(Cl)c2c1C(=O)[C@@]1(O2)C(OC)=CC(=O)C[C@H]1C]

### Example 4: The Synthesis Procedure of 4 Position Cyclopropylmethyl Griseofulvin Derivative (Compound 04)

The different in this procedure is that the bromoalkene is cyclopropylmethyl bromide, and a white solid was obtained. Yield 60%, m.p. 174-176° C.; ¹H NMR (400 MHZ, CDCl₃) δ 6.10 (s, 1H, H-5), 5.53 (s, 1H, H-3′), 4.04-3.89 (m, 5H, —OCH₂—, H-10), 3.62 (s, 3H, H-11), 3.05 (dd, J=16.7, 13.5 Hz, 1H, H-5′), 2.87-2.80 (m, 1H, H-6′), 2.41 (dd, J=16.7, 4.7 Hz, 1H, H-5′), 1.37-1.32 (m, 1H, —CH—C₂H₄), 0.96 (d, J=6.7 Hz, 3H, H-8), 0.76-0.62 (m, 2H, —CH₂—), 0.42-0.38 (m, 2H, —CH₂—). ¹³C NMR (100 MHZ, CDCl₃) δ 197.24 (C-4′), 192.24 (C-3), 171.08 (C-2′), 169.61 (C-7a), 164.50 (C-6), 157.48 (C-4), 105.45 (C-3a), 104.89 (C-3′), 97.12 (C-7), 90.88 (C-5), 90.74 (C-2), 74.42 (—OCH₂—), 57.09 (C-10), 56.78 (C-11), 40.12 (C-5′), 36.65 (C-6′), 14.37 (C-8), 9.92 (—CH—C₂H₄), 3.66 (—CH₂—), 3.62 (—CH₂—). HR-MS (ESI): m/z calcd. for C₂₀H₂₁ClO₆Na: 415.0924; found: 415.0928 [M+Na]⁺.

Chemical structure of compound 04 was shown below.

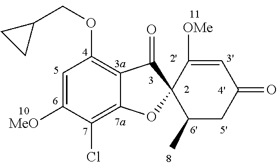 [SMILES: COC1=CC(=O)C[C@@H](C)[C@]12Oc1c(Cl)c(OC)cc(OCC3CC3)c1C2=O]

### Example 5: The Synthesis Procedure of 4 Position Methoxymethyl Griseofulvin Derivative (Compound 05)

The different in this procedure is that the bromoalkene is methoxymethyl bromide, and a white solid was obtained. Yield 67%, m.p.124-125° C.; ¹H NMR (400 MHZ, CDCl₃) δ 6.48 (s, 1H, H-5), 5.55 (s, 1H, H-3′), 5.33 (s, 2H, —OCH₂O—), 4.00 (s, 3H, H-10), 3.63 (s, 3H, H-11), 3.53 (s, 3H, —OCH₃), 3.02 (dd, J=16.5, 13.5 Hz, 1H, H-5′), 2.90-2.81 (m, 1H, H-6′), 2.43 (dd, J=16.6, 4.6 Hz, 1H, H-5′), 0.96 (d, J=6.6 Hz, 3H, H-8). ¹³C NMR (100 MHZ, CDCl₃) δ 197.21 (C-4′), 192.65 (C-3), 170.94 (C-2′), 169.44 (C-7a), 164.53 (C-6), 155.53 (C-4), 105.75 (C-3a), 104.99 (C-3′), 98.31 (C-7), 95.49 (C-5), 93.27 (—OCH₂O—), 90.83 (C-2), 57.24 (C-10), 57.11 (—OCH₃), 56.84 (C-11), 40.11 (C-5′), 36.61 (C-6′), 14.40 (C-8). HR-MS (ESI): m/z calcd. for C₁₈H₁₉ClO₇Na: 405.0717; found: 405.0728 [M+Na]⁺.

Chemical structure of compound 05 was shown below.

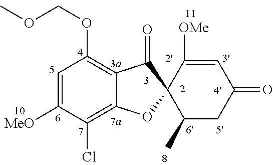 [SMILES: COCOc1cc(OC)c(Cl)c2c1C(=O)[C@@]1(O2)C(OC)=CC(=O)C[C@H]1C]

### Example 6: The Synthesis Procedure of 4 Position 2-Fluorobenzyl Griseofulvin Derivative (Compound 06)

The different in this procedure is that the bromoalkene is 2-fluorobenzyl bromide, and a white solid was obtained. Yield, 83%, m.p. 212-213° C.; ¹H NMR (400 MHZ, CDCl₃) δ 7.71-7.64 (m, 1H, ArH), 7.37-7.29 (m, 1H, ArH), 7.19 (d, J=1.0 Hz, 1H, ArH), 7.12-7.04 (m, 1H, ArH), 6.25 (s, 1H, H-5), 5.54 (s, 1H, H-3′), 5.31 (s, 2H, —OCH₂—), 3.99 (s, 3H, H-10), 3.62 (s, 3H, H-11), 3.04 (dd, J=16.6, 13.5 Hz, 1H, H-5′), 2.92-2.79 (m, 1H, H-6′), 2.43 (dd, J-16.7, 4.7 Hz, 1H, H-5′), 0.96 (d, J=6.7 Hz, 3H, H-8). ¹³C NMR (100 MHZ, CDCl₃) δ 197.08 (C-4′), 192.31 (C-3), 170.92 (C-2′), 169.64 (C-7a), 164.57 (C-6), 161.26 (C-4), 158.82 (C-3a), 156.43 (C-3′), 130.24 (d, J=8.2 Hz, ArC), 129.58 (d, J=3.5 Hz, ArC), 124.97 (d, J=3.7 Hz, ArC), 122.68 (d, J=13.6 Hz, ArC), 115.32 (d, J=21.0 Hz, ArC), 104.97(C-7), 105.99-96.49 (m, ArC), 91.20 (C-5), 90.89 (C-2), 64.55 (d, J=4.5 Hz, —OCH₂—), 57.16 (C-10), 56.80 (C-11), 40.16 (C-5′), 36.62 (C-6′), 14.38 (C-8). HR-MS (ESI): m/z calcd. for C₂₃H₂₀ClFO₆Na: 469.0830; found: 469.0832 [M+Na]⁺.

Chemical structure of compound 06 was shown below.

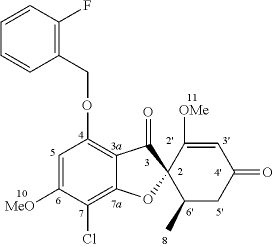 [SMILES: COC1=CC(=O)C[C@@H](C)[C@]12Oc1c(Cl)c(OC)cc(OCc3ccccc3F)c1C2=O]

### Example 7: The Synthesis Procedure of 4 Position 3-Methylthiophene Griseofulvin Derivative (Compound 07)

The different in this procedure is that the bromoalkene is 3-bromomethylthiophene, and a white solid was obtained. Yield, 73%, m.p. 212-214° C.; ¹H NMR (400 MHZ, CDCl₃) δ 7.42-7.39 (m, 1H, —S—CH═), 7.35 (dd, J=5.0, 3.0 Hz, 1H, —S—CH≡CH—), 7.17 (dd, J=5.0, 1.3 Hz, —S—CH—CH—), 6.18 (s, 1H, H-5), 5.54 (s, 1H, H-3′), 5.27 (dd, J=2.6, 1.0 Hz, 2H, —OCH₂—), 3.96 (s, 3H, H-10), 3.62 (s, 3H, H-11), 3.04 (dd, J=16.7, 13.5 Hz, 1H, H-5′), 2.89-2.81 (m, 1H, H-6′), 2.45-2.40 (m, 1H, H-5′), 0.96 (d, J=6.7 Hz, 3H, H-8). ¹³C NMR (100 MHz, CDCl₃) δ 197.15 (C-4′), 192.32 (C-3), 170.95 (C-2′), 169.58 (C-7a), 164.45 (C-6), 156.74 (C-4), 136.41 (—S—CH═), 126.97 (—S—CH≡CH—), 126.42 (—S—CH—CH—), 123.22 (—S—CH—C—), 105.61 (C-3a), 104.94 (C-3′), 97.58 (C-7), 91.42 (C-5), 90.85 (C-2), 67.44 (—OCH₂—), 57.10 (C-10), 56.81 (C-11), 40.12 (C-5′), 36.60 (C-6′), 14.37 (C-8). HR-MS (ESI): m/z calcd. for C₂₁H₁₉ClO₆SNa: 457.0489; found: 457.0488 [M+Na]⁺.

Chemical structure of compound 07 was shown below.

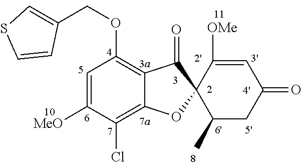 [SMILES: COC1=CC(=O)C[C@@H](C)[C@]12Oc1c(Cl)c(OC)cc(OCc3ccsc3)c1C2=O]

### Example 8: The Synthesis Procedure of 4 Position 1-(4-(Methyl)Phenyl)-Ethanone Griseofulvin Derivative (Compound 08)

The different in this procedure is that the bromoalkene is 1-(4-(Bromomethyl)-phenyl)ethanone, and a white solid was obtained. Yield, 55%, m.p. 275-276° C.; ¹H NMR (400 MHZ, CDCl₃) δ 7.99 (d, J=8.4 Hz, 2H, ArHx2), 7.59 (d, J=8.5 Hz, 2H, ArH×2), 6.16 (s, 1H, H-5), 5.55 (s, 1H, H-3′), 5.31 (s, 2H, —OCH₂—), 3.96 (s, 3H, H-10), 3.63 (s, 3H, H-11), 3.04 (dd, J=16.7, 13.5 Hz, 1H, H-5′), 2.91-2.79 (m, 1H, H-6′), 2.60 (s, 3H, —CH₃), 2.42 (d, J=4.6 Hz, 1H, H-5′), 0.97 (d, J=6.6 Hz, 3H, H-8). ¹³C NMR (100 MHZ, CDCl₃) δ 197.73 (—CO—), 197.01 (C-4′), 192.37 (C-3), 170.84 (C-2′), 169.67 (C-7a), 164.52 (C-6), 156.38 (ArC), 140.71 (C-4), 137.08 (ArC), 128.98 (ArC), 126.80 (ArC), 105.67 (C-3a), 105.01 (C-3′), 97.99 (C-7), 91.43 (C-5), 90.96 (C-2), 70.44 (—OCH₂—), 57.14 (C-10), 56.83 (C-11), 40.15 (C-5′), 36.61 (C-6′), 26.80 (—CH₃), 14.39 (C-8). HR-MS (ESI): m/z calcd. for C₂₅H₂₃ClO₇Na: 493.1030; found: 493.1035 [M+Na]⁺.

Chemical structure of compound 08 was shown below.

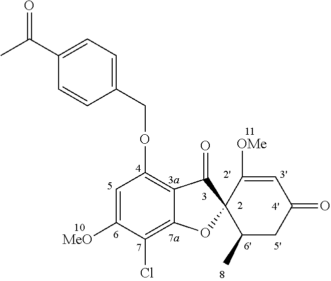 [SMILES: COC1=CC(=O)C[C@@H](C)[C@]12Oc1c(Cl)c(OC)cc(OCc3ccc(C(C)=O)cc3)c1C2=O]

### Example 9: The Synthesis Procedure of 4 Position Cyclopentyl Griseofulvin Derivative (Compound 09)

The different in this procedure is that the bromoalkene is Bromocyclopentane, and a white solid was obtained. Yield, 43%, m.p. 202-204° C.; ¹H NMR (400 MHz, CDCl₃) δ 6.11 (s, 1H, H-5), 5.52 (s, 1H, H-3′), 4.90-4.87 (m, 1H, —OCH—), 3.99 (s, 3H, H-10), 3.61 (s, 3H, H-11), 3.04 (dd, J=16.7, 13.5 Hz, 1H, H-5′), 2.85-2.81 (m, 1H, H-6′), 2.43-2.37 (m, 1H, H-5′), 2.04-1.90 (m, 4H, —CH₂—×2), 1.90-1.77 (m, 2H, —CH₂—), 1.69-1.58 (m, 2H, —CH₂—), 0.95 (d, J=6.7 Hz, 3H, H-8). ¹³C NMR (100 MHZ, CDCl₃) δ 197.23 (C-4′), 192.19 (C-3), 171.16 (C-2′), 169.65 (C-7a), 164.32 (C-6), 156.82 (C-4), 105.69 (C-3a), 104.82 (C-3′), 96.69 (C-7), 91.83 (C-5), 90.67 (C-2), 81.67 (—OCH—), 57.01 (C-10), 56.77 (C-11), 40.10 (C-5′), 36.65 (C-6′), 33.02 (CH₂—), 32.88 (—CH₂—), 24.19 (—CH₂—), 24.17 (—CH₂—), 14.37 (C-8). HR-MS (ESI): m/z calcd. for C₂₁H₂₃ClO₆Na: 429.1081; found: 429.1087 [M+Na]⁺.

Chemical structure of compound 09 was shown below.

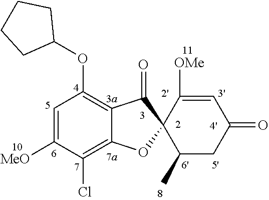 [SMILES: COC1=CC(=O)C[C@@H](C)[C@]12Oc1c(Cl)c(OC)cc(OC3CCCC3)c1C2=O]

### Example 10: Antifungal Activity Assay

The antifungal activities were evaluated against seven plant phytopathogenic fungi, by the mycelial growth inhibitory rate method, according to a reported method (Yu-Bin Bai, Meng Zhang, Ding Li, Yu Zhao, Liang-Zhu Huang, and Jin-Ming Gao. J. Agric. Food. Chem., 2023, 71, 16, 6236-6248). The target phytopathogenic fungi are Cytospora sp., Alternaria solani Sorauer, Alternaria alternate, Botrytis sp., Botrytis cinerea Pers., Colletotrichum gloeosporioides and Botryosphaeria dothidea. 100 μg/mL PDA mediums were prepared at 50° C. The petri dishes were incubated and cultivated at 28° C. for 48-72 h. Each sample was measured in triplicate, each colony diameter of all triplicates was measured 4 times by cross bracketing method. Hymexazol (Hym) as the positive control and the result of inhibition rate was calculated according to the formula:

$$Inhibition rate ( \% ) = ( C - T ) / ( C - 4 mm ) \times 100 \% ,$$

where C represents the diameter of fungal growth on untreated PDA, and T represents the diameter of fungal growth on treated PDA. The results are reported in Table 1. The activity results suggested these 9 griseofulvin derivatives exhibited very excellent antifungal activity.

TABLE 1
Preliminary antifungal activity of compounds at 100 μg/mL

| | Average values of antifungal rate (%) | | | | | | |
|---|---|---|---|---|---|---|---|
| Compd | C.s. | A.s. | A.a. | B.s. | B.c. | C.g. | B.d. |
| 01 | 96.62 | 100.00 | 97.41 | 100.00 | 98.61 | 91.34 | 97.99 |
| 02 | 52.70 | 77.05 | 49.14 | 85.23 | 56.94 | 70.08 | 51.68 |
| 03 | 29.05 | 26.92 | 7.41 | 62.26 | 19.05 | 68.42 | 31.75 |
| 04 | 65.54 | 70.49 | 41.38 | 69.80 | 50.69 | 48.03 | 26.17 |
| 05 | 73.65 | 75.41 | 49.14 | 88.59 | 65.97 | 71.65 | 58.39 |
| 06 | 6.08 | 13.11 | 3.45 | 69.80 | 55.56 | 75.59 | 18.12 |
| 07 | 54.73 | 62.30 | 43.10 | 73.83 | 59.72 | 72.44 | 42.28 |
| 08 | 7.43 | 0.00 | 2.59 | 47.65 | 43.06 | 25.98 | 4.70 |
| 09 | 59.46 | 45.90 | 31.03 | 63.09 | 59.72 | 40.94 | 34.23 |
| Hym | 48.04 | 97.44 | 92.59 | 92.45 | 100.00 | 43.16 | 55.56 |

Note:
C.s., Cytospora sp.;
A.s., Alternaria solani Soraue;
A.a., Alternaria alternate;
B.s., Botrytis sp.;
B.c., Botrytis cinerea Pers.;
C.g., Colletotrichum gloeosporioides;
B.d., Botryosphaeria dothidea;
Hym., Hymexazol.

The compounds with inhibition rates superior to 70% at 100 μg/mL were chosen. PDA mediums with different compound concentrations (100, 50, 25, 12.5, 6.25, 3.125, 1.5625, 0.78125, 0.3906, 0.1953, 0.0977, 0.0488 μg/mL) was prepared and their half inhibitory concentration (IC₅₀) values were accurately tested. All half inhibitory concentration results are shown in Table 2 and Hymexazol (Hym) as the positive control.

TABLE 2
The half inhibitory concentration values of antifungal activity

| | IC₅₀ ± SD/(μg/mL) | | | | | | |
|---|---|---|---|---|---|---|---|
| Compd | C.s. | A.s. | A.a. | B.s. | B.c. | C.g. | B.d. |
| 01 | 1.84 ± 0.01 | 1.32 ± 0.01 | 1.28 ± 0.00 | 2.84 ± 0.22 | 2.04 ± 0.12 | 0.06 ± 0.02 | 1.35 ± 0.07 |
| 02 | >50 | 14.87 ± 5.66 | >100 | 3.93 ± 0.80 | >50 | 4.05 ± 0.46 | >50 |
| 03 | >100 | >100 | >100 | >25 | >100 | >25 | >100 |
| 04 | >50 | 17.85 ± 0.04 | >25 | >100 | >100 | >100 | >100 |
| 05 | 81.41 ± 1.23 | 83.21 ± 14.50 | >50 | 31.56 ± 7.30 | >25 | 16.15 ± 1.30 | >25 |
| 06 | >100 | >100 | >100 | >25 | >50 | 1.92 ± 0.19 | >100 |
| 07 | >50 | >50 | >100 | 17.58 ± 0.27 | >25 | 3.82 ± 0.09 | >50 |
| 08 | >100 | >100 | >100 | >100 | >100 | >100 | >100 |
| 09 | >25 | >100 | >100 | >25 | >25 | >100 | >100 |
| Hym | >100 | 45.29 ± 14.04 | 15.58 ± 0.13 | 13.48 ± 1.89 | >6.25 | >100 | >50 |

The activity results suggested that compared with the positive control hymexazol and thifluzamide, these 9 griseofulvin derivatives showed very excellent antifungal activity against phytopathogenic fungi. To our surprise, compound 01 exhibited significant and extensive antifungal activity against all these seven target fungi, and their IC₅₀ values are respectively 1.84±0.01 μg/mL, 1.32±0.01 μg/mL, 1.28±0.00 μg/mL, 2.84±0.22 μg/mL, 2.04±0.12 μg/mL, 0.06±0.02 μg/mL, 1.35±0.07 μg/mL, particularly against Colletotrichum gloeosporioides (IC₅₀=0.06±0.02 μg/mL). Furthermore, compounds 02, 06 and 07 also showed remarkable activity against Colletotrichum gloeosporioides, their IC₅₀ values are respectively 4.05±0.46 μg/mL, 1.92±0.19 μg/mL, 3.82±0.09 μg/mL. Compound 02 showed excellent activity against Botrytis sp. (IC₅₀=3.93±0.80 μg/mL).

In present disclosure, the compounds (griseofulvin 4 position etherified derivatives) can be used to be fungicide against phytopathogenic fungi. These compounds can used to as main activity components which could be prepared to be some emulsion, hydrating agent, flowable agent, aqueous solution or their mixture as a preventive or direct antifungal agent against phytopathogenic fungi.

1. Griseofulvin 4 position etherified derivatives, wherein chemical structures of the griseofulvin 4 position etherified derivatives are shown in a formula I below.

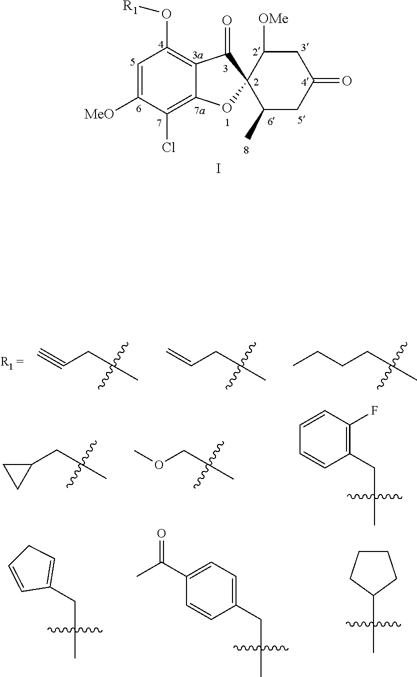

2. The preparation method of the griseofulvin 4 position etherified derivatives as claimed in claim 1, comprising:
with organic solvent and base, intermediate II reacted with different bromoalkanes, griseofulvin 4 position etherified derivatives were obtained;
wherein a chemical structure of the intermediate II is expressed as follows.

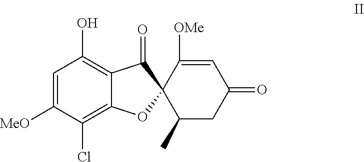 [SMILES: COC1=CC(=O)C[C@@H](C)[C@]12Oc1c(Cl)c(OC)cc(O)c1C2=O]

3. The preparation method as claimed in claim 2, wherein after the reacting is finished, a reaction mixture is obtained, and the preparation method specifically comprises: concentrating the reaction mixture and then purifying the reaction mixture after the concentrating by a column chromatography method, to thereby obtain the griseofulvin 4 position etherified derivatives.

4. The preparation method as claimed in claim 2, wherein the organic solvent is but not limited to acetone or THF or their mixture.

5. The preparation method as claimed in claim 2, wherein the base is but not limited to K₂CO₃ or Na₂CO₃ or Cs₂CO₃.

6. The application of the Griseofulvin 4 position etherified derivatives as claimed in claim 1, comprising: using the Griseofulvin 4 position etherified derivative as a preventive or direct antifungal agent against phytopathogenic fungi.

7. The application as claimed in claim 6, wherein the phytopathogenic fungi includes one or at least two selected from the group consisting of Cytospora sp., Alternaria solani Sorauer, Alternaria alternate, Botrytis sp., Botrytis cinerea Pers., Colletotrichum gloeosporioides, and Botryosphaeria dothidea.
